(19) United States
(12) Patent Application Publication
(10) Pub. No.: US 2026/0157618 A1
(43) Pub. Date: Jun. 11, 2026

(54) GUIDE STRUCTURE AND SPINAL ENDESCOPE ASSEMBLY USING THE SAME

(71) Applicants: YEN-TSUNG LIN, Taipei City (TW); SHIH-HAO CHENG, Taipei City (TW)

(72) Inventors: YEN-TSUNG LIN, Taipei City (TW); SHIH-HAO CHENG, Taipei City (TW)

(21) Appl. No.: 18/973,124

(22) Filed: Dec. 9, 2024

(51) Int. Cl. A61B 1/313; A61B 1/00; A61B 17/34

(52) U.S. Cl. CPC A61B 1/3135; A61B 1/00154; A61B 17/3468

(57) ABSTRACT

The present invention relates to a guide structure and a spinal endoscope assembly using the same. The guide structure includes an upper holder, a conduit, and a transparent tube. The spinal endoscope assembly includes the guide structure and a spinal gun. After the guide structure is inserted into the patient's body, the spinal gun can enter the patient's body through a perforation, a guide hole, and a through hole of the guide structure, providing medical personnel with a means to operate. A camera module is used for imaging and can search in all directions to observe the affected area without obstruction, owing to a transparent design applied in an observation portion of the transparent tube. This not only saves time during surgery but also ensures patient safety by preventing additional harm to the affected area.

### BACKGROUND OF THE INVENTION

### Fields of the Invention

The present invention relates to a guide structure and a spinal endoscope assembly using the same and, particularly to, a technique applied to the field of medical instruments.

### Descriptions of Related Art

In endoscopic surgery, medical personnel typically make incisions on both sides of the spine to establish surgical channels. To allow the endoscope to enter, the channels must be expanded to ensure instruments can smoothly reach the targeted surgical site. Then, medical personnel can insert a tubular instrument from the incision site. This tubular instrument, as shown in FIGS. 15 and 16, primarily includes a handheld component 9 and a fixed tube 10 running through the handheld component 9. The fixed tube 10 is a metal tube with a slanted opening at the end away from the handheld component 9. Medical personnel can hold the handheld component 9 and insert the slanted end of the fixed tube 10 into the affected area. Afterward, the endoscope 20 is inserted through an opening on one end of the fixed tube 10, near the point where the fixed tube 10 passes through the handheld component 9, for viewing the area to be treated.

However, the aforementioned surgical procedure, though commonly employed today, often encounters issues resulting from the fixed tube 10 being made of metal. Since the inserted endoscope 20 is often positioned incorrectly for viewing, it may require rotation. However, rotating the endoscope 20 can obstruct the view with part of the fixed tube 10, requiring medical personnel to rotate and adjust the fixed tube 10. This not only shows disrespect to the patient but may also cause additional wounds. More critically, continuous rotation may still fail to provide a clear view for the endoscope 20, leading to severe medical errors. For medical personnel, every second counts during surgery, and unnecessary delays caused by repeatedly rotating the tubular instrument extend the surgery time, increasing the psychological pressure on medical personnel.

Additionally, the lens part of the endoscope 20 generally faces forward. Once the endoscope 20 is inserted into the fixed tube, the viewable range is limited by the diameter of the fixed tube 10. Besides the the obstruction mentioned earlier, the viewing range is significantly restricted.

### SUMMARY OF THE INVENTION

The primary objective of the present invention is to enable medical personnel to operate the endoscope through a tubular instrument without being obstructed by a fixed member, allowing for an all-directional search for the area to be viewed. This not only saves time during the surgery but also ensures the safety of the patient by preventing further injury to the affected area, thereby addressing the shortcomings of tubular instruments in the prior art.

To achieve the above-mentioned effects and address the shortcomings of the prior art, the present invention provides a guide structure and a spinal endoscope assembly using the guide structure. The guide structure of the present invention includes: an upper holder, having a perforation extending from a top end to a bottom end of the upper holder; a conduit, having a first end for being inserted into and tightly connected to an opening of the perforation of the upper holder and a guide hole extending from a top end to a bottom end of the conduit and configured in communication with the perforation; and a transparent tube, sleeved around a second end of the conduit, wherein the transparent tube includes a fitting portion, an observation portion, and a through hole extending through the fitting portion and the observation portion, the fitting portion being sleeved around the conduit and the observation portion being designed to be transparent.

In the application of the aforementioned guide structure, the present invention provides a spinal endoscope assembly, which comprises: a guide structure, including: an upper holder, having a perforation extending from a top end to a bottom end of the upper holder; a conduit, having a first end for being inserted into and tightly connected to an opening of the perforation of the upper holder and a guide hole extending from a top end to a bottom end of the conduit and configured in communication with the perforation; and a transparent tube, sleeved around a second end of the conduit, wherein the transparent tube includes a fitting portion, an observation portion, and a through hole extending through the fitting portion and the observation portion, the fitting portion being sleeved around the conduit and the observation portion being designed to be transparent; and a spinal gun, including a gun body and an endoscope tube, wherein a first end of the endoscope tube is insertable into the gun body, a camera module is installed inside the endoscope tube and has a portion exposed from a second end of the endoscope tube and positioned at an angle relative to the endoscope tube; the second end of the endoscope tube, which reveals the camera module, is insertable into the perforation of the upper holder, through the guide hole of the conduit, and out of the through hole of the transparent tube for imaging purposes.

### BRIEF DESCRIPTION OF THE DRAWINGS

FIG. 1 is a perspective schematic view of the guide structure in the present invention.

FIG. 2 is an exploded perspective schematic view of FIG. 1.

FIG. 3 is a cross-sectional schematic view taken along line III-III in FIG. 1.

FIG. 4 is a perspective schematic view of the spinal gun in the present invention.

FIG. 5 is a partial exploded schematic view of FIG. 4.

FIG. 5A is another partial exploded schematic view of FIG. 4.

FIG. 6 is a cross-sectional schematic view taken along line VI-VI in FIG. 4.

FIG. 6A is a cross-sectional schematic view taken along line VIA-VIA in FIG. 4.

FIG. 7 is a perspective schematic view of the use state of the guide structure being inserted into the affected area of a body in accordance with the present invention.

FIG. 8 is a cross-sectional schematic view taken along line VIII-VIII in FIG. 7.

FIG. 9 is a perspective schematic view of the endoscope tube of the spinal gun being inserted into the guide structure in accordance with the present invention.

FIG. 10 is a cross-sectional schematic view taken along line X-X in FIG. 9.

FIG. 11 is a partially enlarged schematic view of FIG. 10.

FIG. 12 is a schematic view of the use state of the present invention in electrical connection with external electronic components.

FIG. 13 is a schematic view of the use state of the two handle sleeves of the spinal gun in the present invention clamping the wiring.

FIG. 14 is another schematic view of the use state of the two handle sleeves of the spinal gun in the present invention clamping the wiring.

FIG. 15 is a perspective schematic view of a conventional tubular instrument.

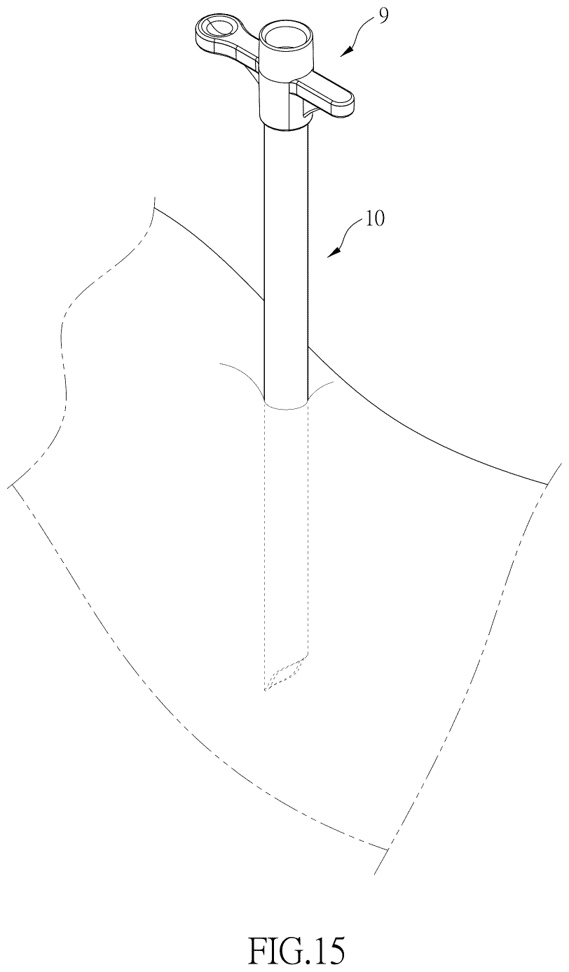

FIG. 16 is an illustration showing the blockage of imaging when the endoscope is inserted into the conventional tubular instrument.

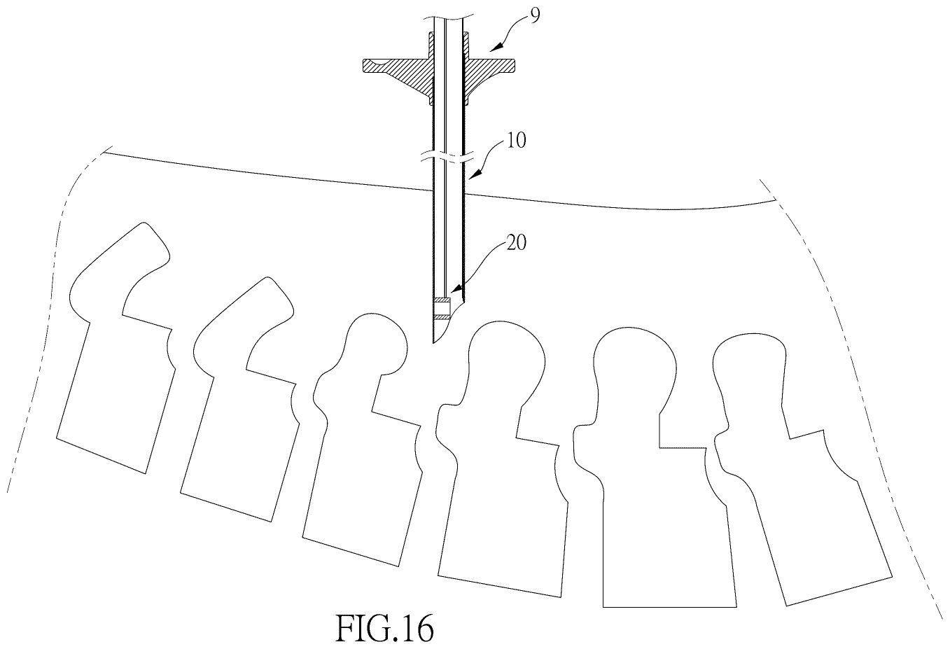

### DETAILED DESCRIPTION OF THE PREFERRED EMBODIMENT

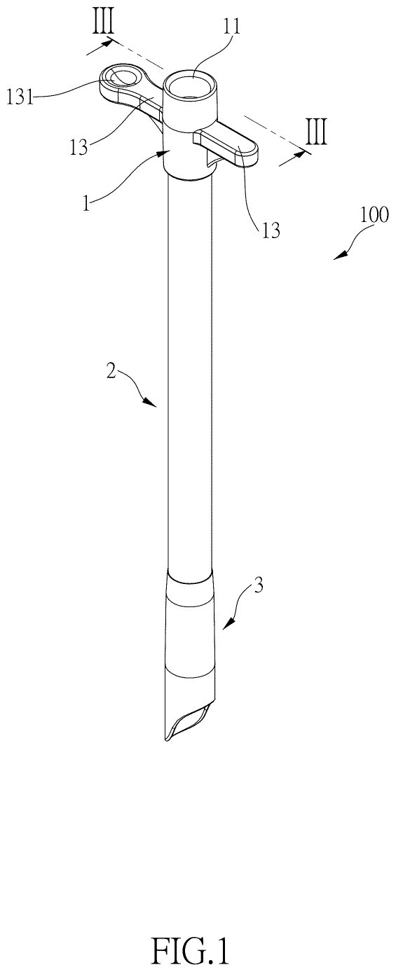

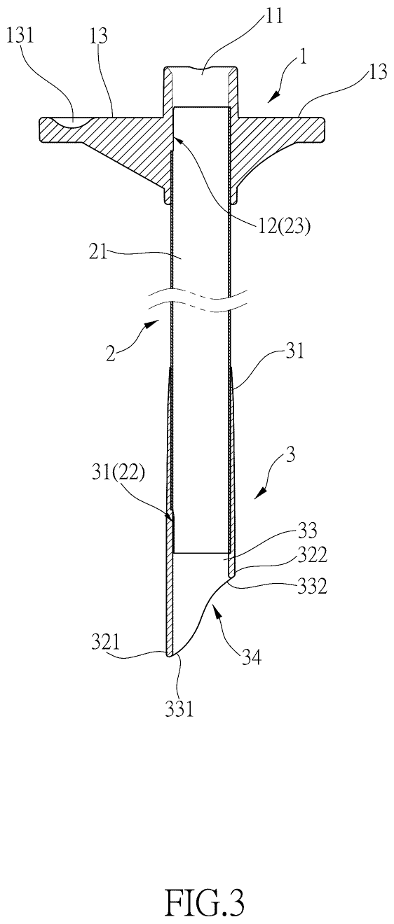

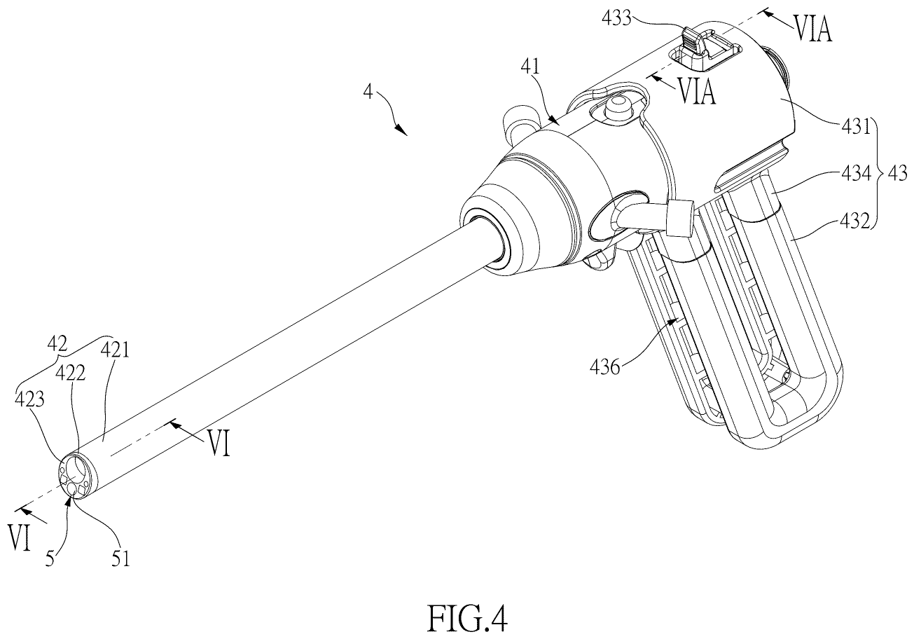

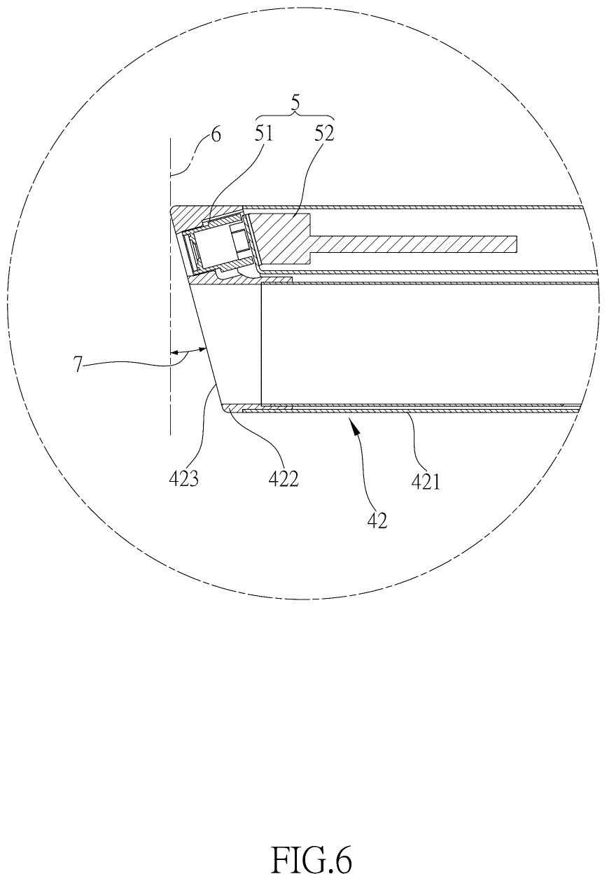

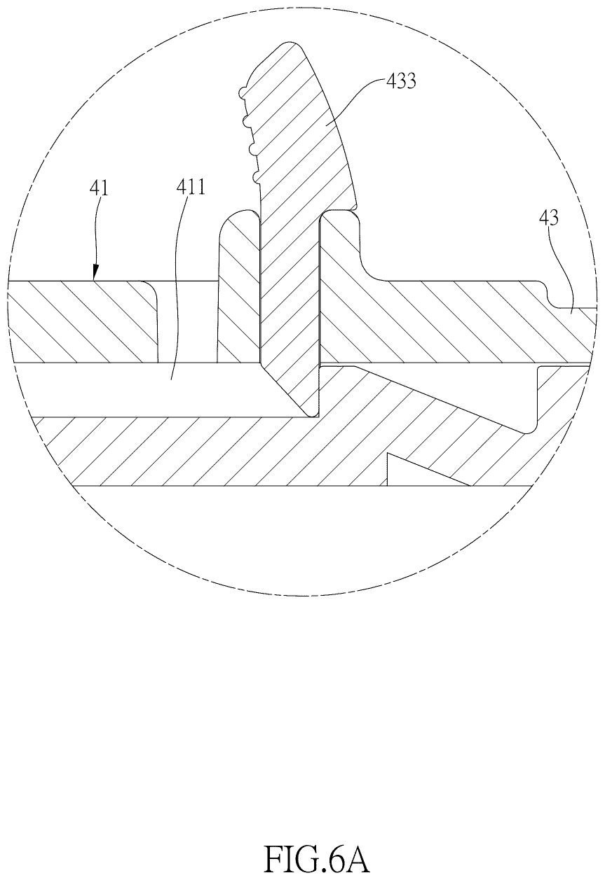

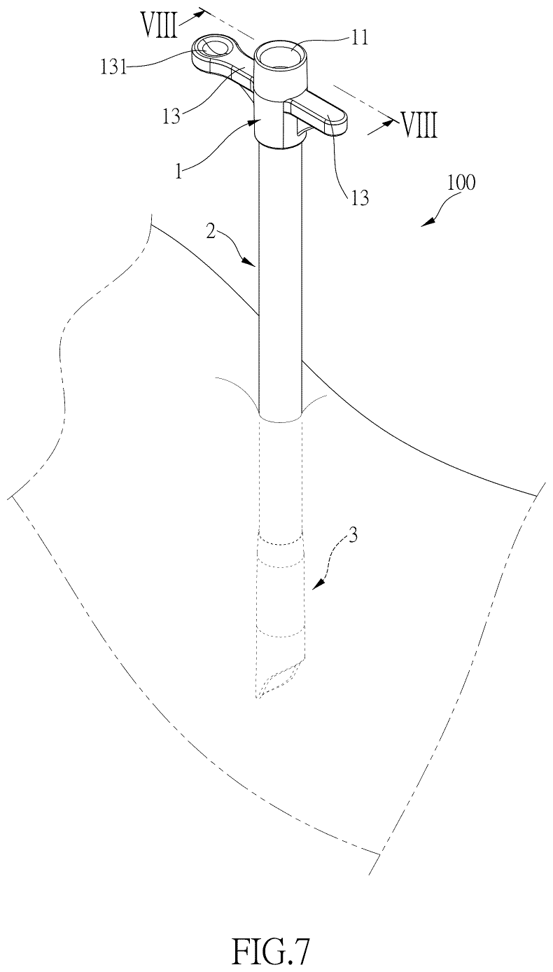

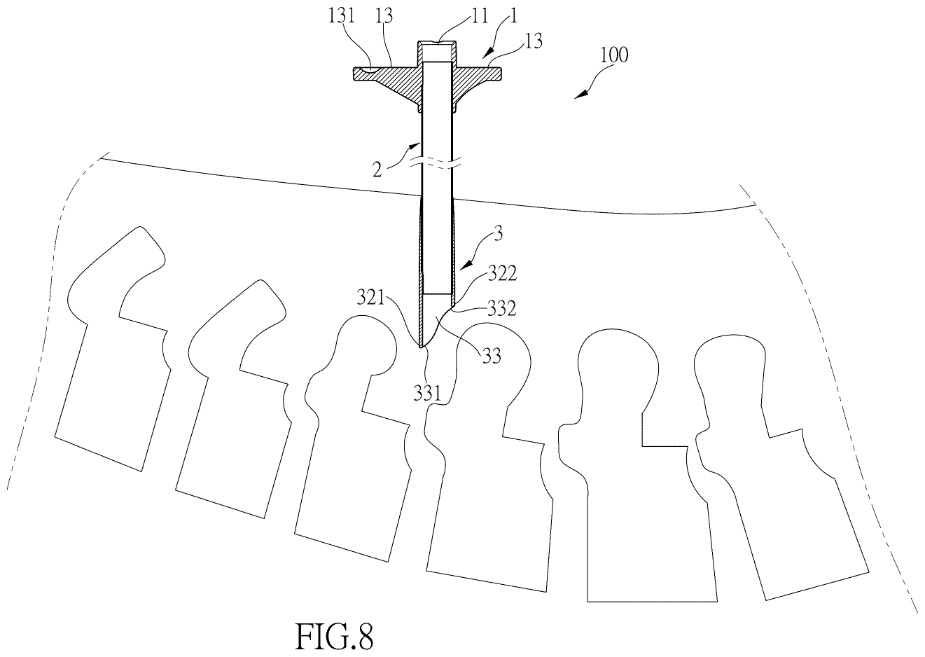

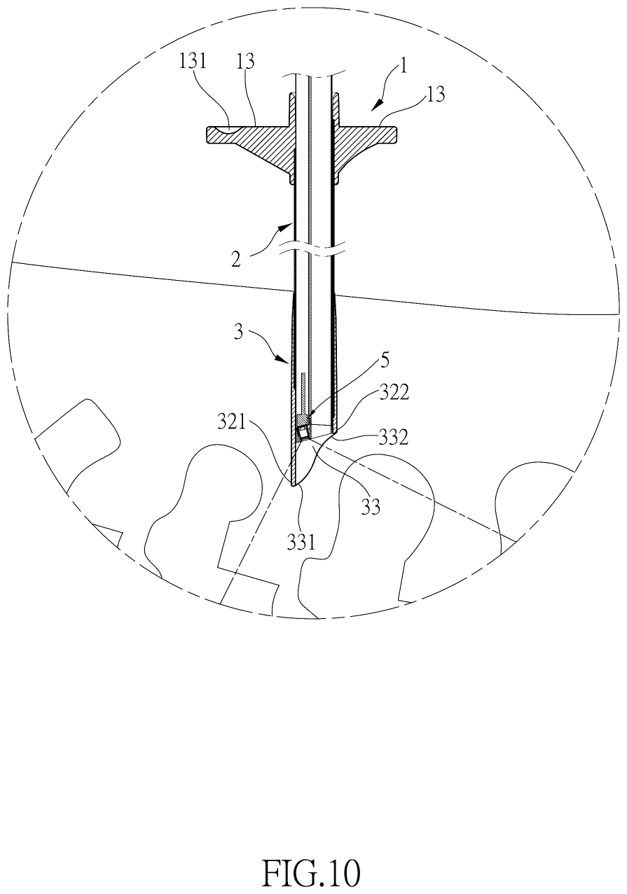

Referring to FIGS. 1 to 12, the guide structure 100 of the present invention includes: an upper holder 1, which has a perforation 11 extending from the top end to the bottom end of the upper holder 1; a conduit 2 in a cylindrical hollow configuration, with a first end inserted into and tightly connected to the opening of the perforation 11 on one side of the upper holder 1 and a guide hole 21 extending from the top end to the bottom end of the conduit 2 and configured in communication with the perforation 11; and a transparent tube 3 in a cylindrical hollow configuration, sleeved around a second end of the conduit 2. The transparent tube 3 includes a fitting portion 31, an observation portion 32, and a through hole 33 extending through both the fitting portion 31 and the observation portion 32. The fitting portion 31 is sleeved around the conduit 2. The observation portion 32 is designed to be transparent. Next, the medical instrument used in conjunction with the guide structure 100 is described. The present invention also provides a spinal endoscope assembly 200, which further includes a spinal gun 4 in addition to the upper holder 1, the conduit 2 and the transparent tube 3, as mentioned above. The spinal gun 4 includes a gun body 41 and an endoscope tube 42, with a first end of the endoscope tube 42 inserted into the gun body 41. Inside the gun body 41, there are components for detection, measurement, and suction pathways for cleaning during surgery. Additionally, a camera module 5 is installed inside the endoscope tube 42 with a portion of the camera module 5 exposed from a second end of the endoscope tube 42. The exposed portion of the camera module 5 is positioned at an angle relative to the endoscope tube. The second end of the endoscope tube 42, which reveals the camera module 5, can be inserted into the perforation 11 of the upper holder 1, through the guide hole 21 of the conduit 2, and out of the through hole 33 of the transparent tube 3 for imaging purposes.

Based on the above configuration of the guide structure 100 and the spinal gun 4 according to the present invention, when medical personnel plan to use the endoscope to view the affected area of the patient's spine, they must first make a small incision in the patient's body, followed by inserting the end, configured with the transparent tube 3, of the guide structure 100 through the incision into the patient's body. After the transparent tube 3 is inside the body, the spinal gun 4 is taken, and the second end of the endoscope tube 42, which reveals the camera module 5, is inserted through the perforation 11 of the upper holder 1 and moved along the conduit 2 through the guide hole 21 until the exposed portion of the camera module 5 reaches the opening of the through hole 33 of the transparent tube 3. Since the observation portion 32 of the transparent tube 3 is designed to be transparent, if the medical personnel do not have a clear view of the affected area through the camera module 5, they can simply adjust the positioning of the spinal gun 4 to change the viewing direction of the camera module 5, allowing a clear view through the observation portion 32 without obstruction. Compared to the issues listed in prior art, where the view is blocked by the tubular instrument, the transparent design of the observation portion 32 in the present invention allows the camera module 5 to capture the affected area inside the transparent tube 3 without blind spots. As a result, the affected area of the patient does not suffer additional injury, and the time taken by medical personnel to locate the affected area is also reduced. This shows that the present invention is an advancement over the prior art.

In addition to the aforementioned definitions of the guide structure 100 and spinal gun 4 in the present invention, the following further details other technical and structural features of the present invention. First, referring to FIGS. 1 to 3 and 7 to 11, in general, for endoscopic surgeries near the spine, medical personnel need to smoothly insert the guide structure 100 into the body (since the human body contains muscles, nerves, etc., external instruments inserted into the body will naturally encounter obstructions and resistance). Therefore, one end of the guide structure 100, which is to be inserted into the body, must function similarly to a tool for breaking open. Accordingly, the observation portion 32 is further cut and shaped from one side to form an indentation 34 at the location corresponding to the opening of the through hole 33. The indentation 34 causes the observation portion 32 to form a lower cutting section 321 and an upper shallow section 322. Additionally, the indentation 34 causes the opening of the through hole 33 to form a lower edge 331 and an upper edge 332. Medical personnel can utilize the lower cutting section 321 and the upper shallow section 322, which form a needle-like shape, to easily break through the body's muscles and nerves, thereby smoothly inserting the observation portion 32 into the affected area and allowing the endoscope tube 42 to capture images successfully.

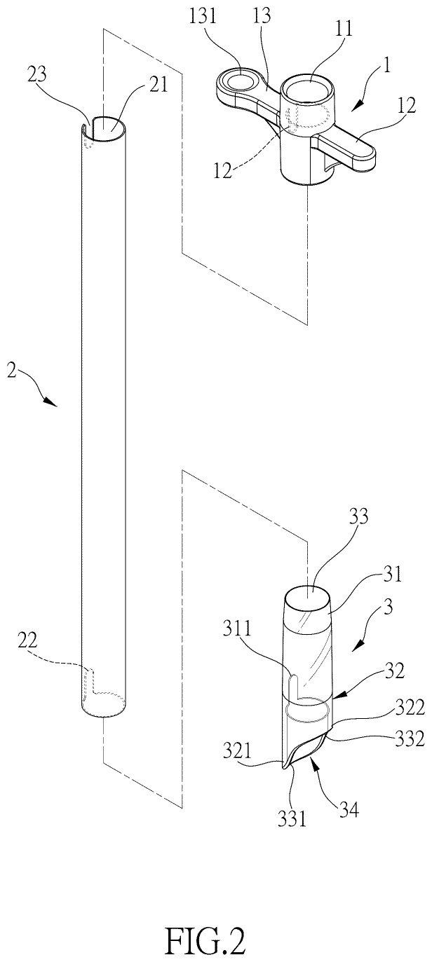

Continuing the above explanation, in order to prevent accidents (such as detachment of the transparent tube 3) when inserting the guide structure 100 into the patient's body or to avoid the separation between the upper holder 1 and the conduit 2 during operation, as shown in FIG. 2, the second end of the conduit 2, designed for insertion into the fitting portion 31 of the transparent tube 3, is further cut with a positioning groove 22, whereas the inner wall of the fitting portion 31 is provided with a coupling rib 311 at the location corresponding to the lower positioning groove 22. When the transparent tube 3 is sleeved around the conduit 2 via the fitting portion 31, the lower positioning groove 22 is aligned with and engaged with the coupling rib 311, ensuring a tight and secure combination between the conduit 2 and the transparent tube 3. Furthermore, the engagement between the lower positioning groove 22 and the coupling rib 311 secures the combination between the conduit 2 and the transparent tube 3. Similarly, the first end of the conduit 2, designed for insertion into the upper holder 1, is further cut with an upper positioning groove 23, whereas the inner wall of the perforation 11 in the upper holder 1 is provided with a coupling rib 12 at the location corresponding to the upper positioning groove 23. When the conduit 2 is inserted into the upper holder 1, the upper positioning groove 23 is aligned with and engaged with the coupling rib 12. The engagement between the upper positioning groove 23 and the coupling rib 12 secures the combination between the upper holder 1 and the conduit 2. This configuration prevents the separation between the upper holder 1 and the conduit 2 when medical personnel operate to remove the guide structure 100, thereby ensuring the safety of medical procedures.

Additionally, to facilitate the smooth insertion of the guide structure 100 into the patient's body and to allow the application of a certain amount of force during guided insertion, the outer side of the upper holder 1 is further extended with two grip handles 13. These two grip handles 13 extend in opposite directions, and one of the grip handles 13 has an end with a groove 131 recessed into the top surface thereof. The design of the groove 131 primarily allows the operator's thumb to press against the groove 131 to apply force. This enables the operator to easily apply force while holding, thereby facilitating the insertion of the transparent tube 3 into the body, as shown in FIG. 1.

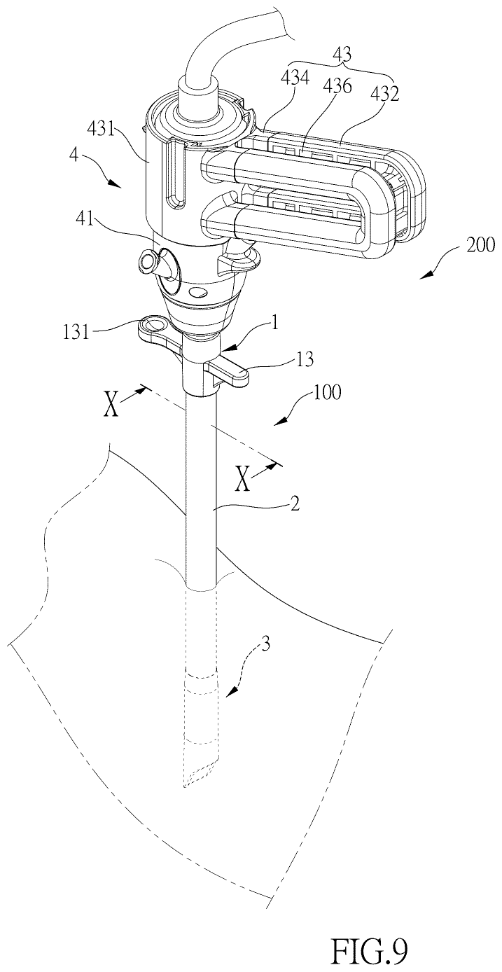

Continuing with the explanation of the spinal gun 4 used for imaging, as shown in FIGS. 9 to 12, to provide better viewing angles for the camera module 5 of the endoscope tube 42, the camera module 5 is positioned to work with the straight cylindrical design of the conduit 2, enabling imaging at a non-direct angle. Structurally, the endoscope tube 42 includes a main tube 421 and an end cap 422. One end of the main tube 421 is connected to the gun body 41, while one end of the end cap 422 is attached to the other end of the main tube 421. The end surface at the other end of the end cap 422 is designed as a slanted face 423, so that one end of the camera module 5 is exposed from and aligned with the slanted face 423. Specifically, a main axis 6 is defined relative to the slanted face 423, and the angle 7 between the slanted face 423 and the main axis 6 ranges from 10 degrees to 35 degrees. From the above, it is evident that the imaging of the camera module 5 depends on the direction of the slanted face. Thus, when the endoscope tube 42 enters the guide structure 100 through the upper holder 1 and reaches the transparent tube 3, the camera module 5 can image at a slanted angle without being obstructed by the inner wall of the through hole 33 of the transparent tube 3. Although the observation portion 32 is designed to be transparent, the obstructions encountered when imaging at a slanted angle are less than those encountered when imaging vertically.

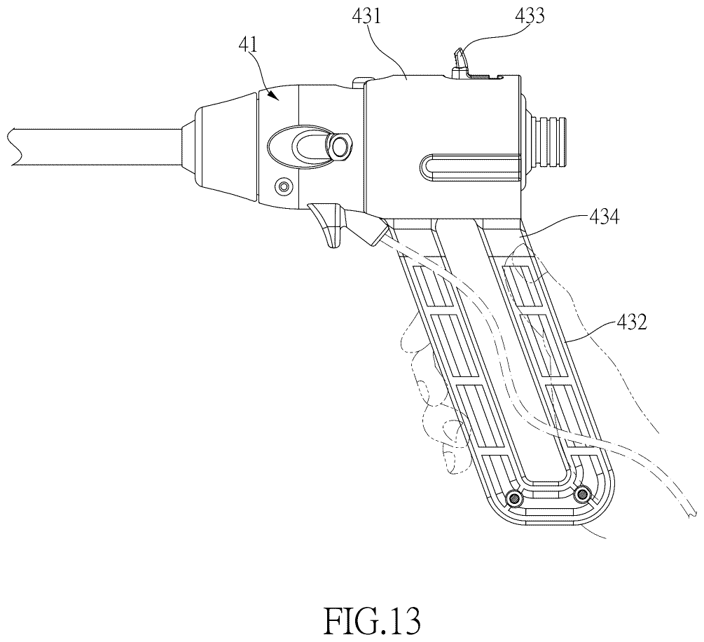

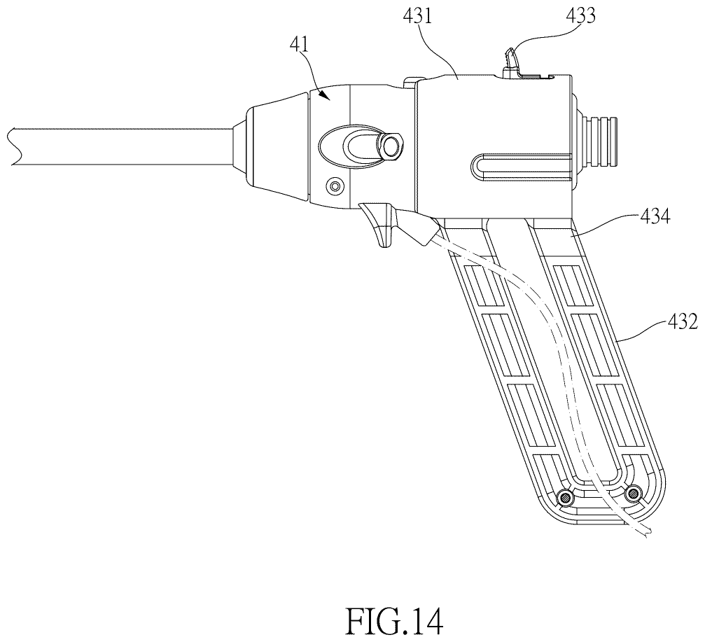

Additionally, to ensure the spinal gun 4 is easy and convenient for medical personnel to handle, an outer casing 43 is further sleeved around the outside of the gun body 41. The outer casing 43 is used depending on the patient's posture. It may not be needed when the patient is lying flat, but in a seated position, holding the gun body 41 may present an angle issue, and the outer casing 43 provides a better grip for medical personnel. The outer casing 43 includes a first sleeve 431 and two handle sleeves 432. The first sleeve 431 is cylindrical in shape, designed to fit around the outer surface of the gun body 41, and is equipped with an elastic buckle 433 that can detachably engage with any of the plurality of grooves 411 on the outer surface of the gun body 41. As such, medical personnel can easily disengage the first sleeve 431 from the gun body 41 by releasing the elastic buckle 433. Furthermore, the first sleeve 431 is equipped with a plurality of connecting columns 434 at the end opposite the elastic buckle 433. The bottom end of each connecting column 434 is recessed inward, forming an assembly groove 435. There are four connecting columns 434 in total. The two handle sleeves 432 are U-shaped, and each has two ends detachably inserted into the assembly grooves 435 of the connecting columns 434 (one handle sleeve 432 has two ends that are detachably inserted into the assembly grooves 435 of two connecting columns 434, forming a grip configuration). The combination of the handle sleeves 432 and the connecting columns 434 creates a clamping area 436, which is primarily used to hold and secure the wiring (electrical wires and water lines) of the gun body 41, as shown in FIGS. 13 and 14.

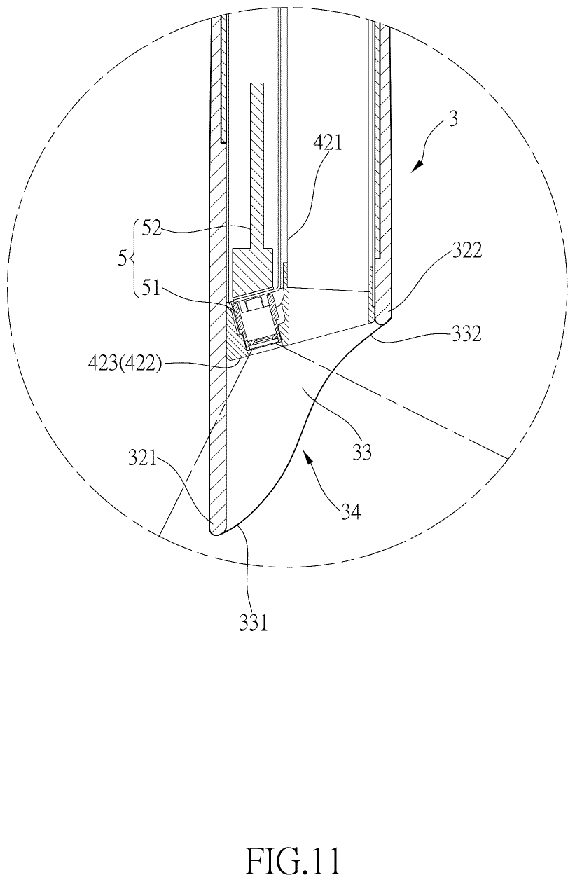

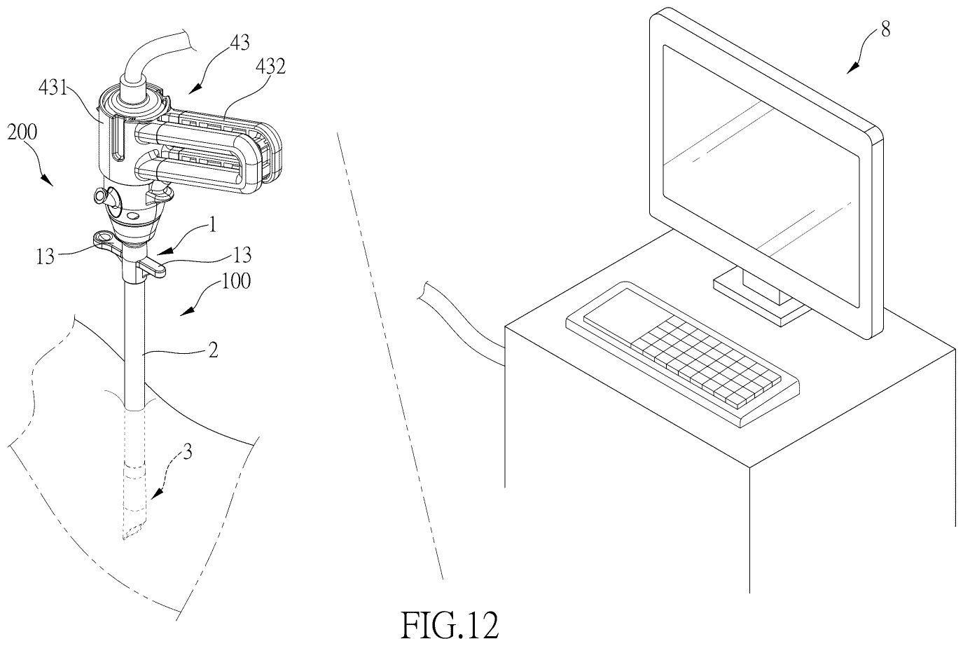

Finally, to make it easier for medical personnel to continuously monitor the imaging results from the camera module 5, the camera module 5 further includes a lens 51 and a chip 52. The chip 52 is located inside the main tube 421, while the lens 51 is positioned inside the end cap 422 and is electrically connected to the chip 52. Further, the chip 52 is electrically connected to an external electronic component 8, either wirelessly or via a wired connection, to transmit the data captured by the lens 51. In this way, medical personnel can inspect the imaging through the external electronic component 8, allowing them to carefully search for the affected area without wasting time blindly searching. This improves efficiency and saves valuable time during the medical procedure, as shown in FIGS. 11 and 12.

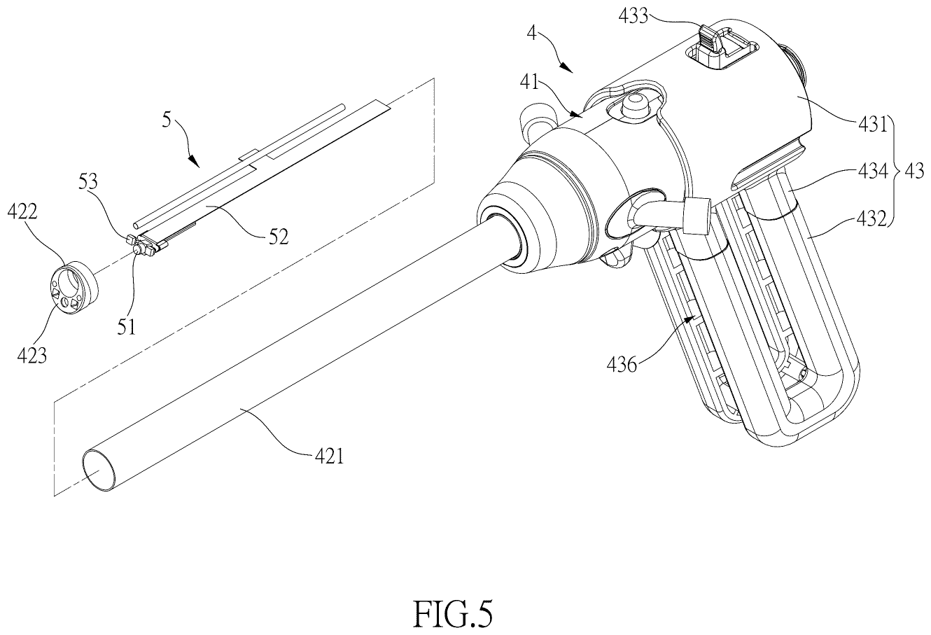

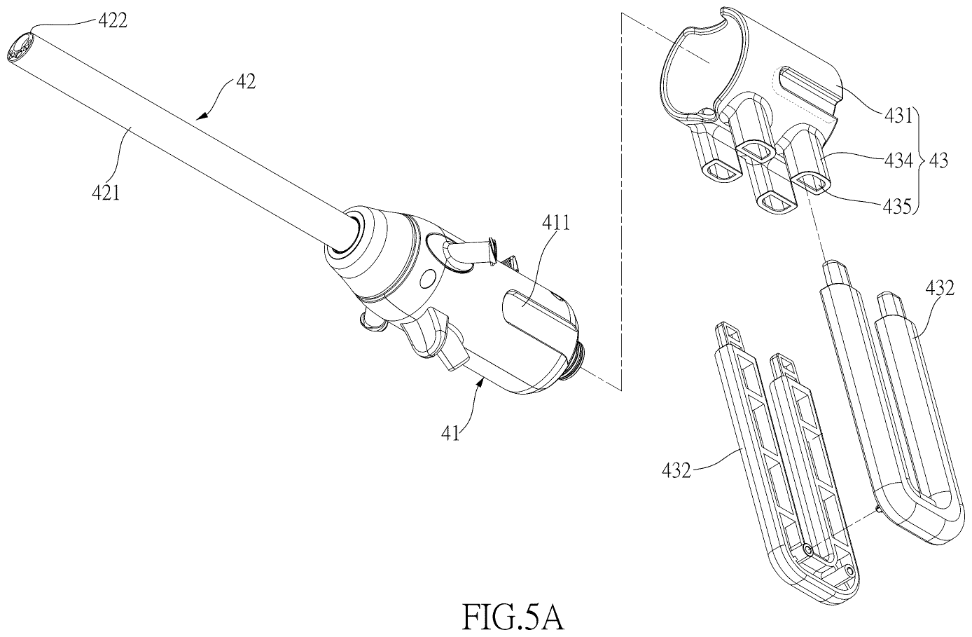

Additionally, to enhance the clarity of captured images for the affected area, at least one light emitting element 53 is further disposed inside the end cap 422 and is electrically connected to the chip 52, as shown in FIG. 5. During medical procedures, the user can utilize the illumination from the light emitting element 53 to increase the brightness for capturing images with the lens 51, allowing the affected area to be observed with greater clarity.

What is claimed is:

1. A guide structure, comprising:
an upper holder, having a perforation extending from a top end to a bottom end of the upper holder;
a conduit, having a first end for being inserted into and tightly connected to an opening of the perforation of the upper holder and a guide hole extending from a top end to a bottom end of the conduit and configured in communication with the perforation; and
a transparent tube, sleeved around a second end of the conduit, wherein the transparent tube includes a fitting portion, an observation portion, and a through hole extending through the fitting portion and the observation portion, the fitting portion being sleeved around the conduit and the observation portion being designed to be transparent.

2. The guide structure as claimed in claim 1, wherein the observation portion is further cut and shaped from one side thereof to form an indentation at a location corresponding to an opening of the through hole; the indentation causes the observation portion to form a lower cutting section and an upper shallow section; and the indentation causes the opening of the through hole to form a lower edge and an upper edge.

3. The guide structure as claimed in claim 1, wherein the second end of the conduit, designed for insertion into the fitting portion of the transparent tube, is further cut with a positioning groove; an inner wall of the fitting portion is provided with a coupling rib at a location corresponding to the lower positioning groove; when the transparent tube is sleeved around the conduit via the fitting portion, the lower positioning groove is aligned with and engaged with the coupling rib; and an engagement between the lower positioning groove and the coupling rib secures a combination between the conduit and the transparent tube.

4. The guide structure as claimed in claim 1, wherein the first end of the conduit, designed for insertion into the upper holder, is further cut with an upper positioning groove; an inner wall of the perforation in the upper holder is provided with a coupling rib at a location corresponding to the upper positioning groove; when the conduit is inserted into the upper holder, the upper positioning groove is aligned with and engaged with the coupling rib; and an engagement between the upper positioning groove and the coupling rib secures a combination between the upper holder and the conduit.

5. The guide structure as claimed in claim 1, wherein an outer side of the upper holder is further extended with two grip handles; the two grip handles extend in opposite directions; and one of the grip handles has an end with a groove recessed into a top surface thereof.

6. A spinal endoscope assembly, comprising:
a guide structure, including:
an upper holder, having a perforation extending from a top end to a bottom end of the upper holder;
a conduit, having a first end for being inserted into and tightly connected to an opening of the perforation of the upper holder and a guide hole extending from a top end to a bottom end of the conduit and configured in communication with the perforation; and
a transparent tube, sleeved around a second end of the conduit, wherein the transparent tube includes a fitting portion, an observation portion, and a through hole extending through the fitting portion and the observation portion, the fitting portion being sleeved around the conduit and the observation portion being designed to be transparent; and
a spinal gun, including a gun body and an endoscope tube, wherein a first end of the endoscope tube is insertable into the gun body, a camera module is installed inside the endoscope tube and has a portion exposed from a second end of the endoscope tube and positioned at an angle relative to the endoscope tube; the second end of the endoscope tube, which reveals the camera module, is insertable into the perforation of the upper holder, through the guide hole of the conduit, and out of the through hole of the transparent tube for imaging purposes.

7. The spinal endoscope assembly as claimed in claim 6, wherein the observation portion is further cut and shaped from one side thereof to form an indentation at a location corresponding to an opening of the through hole; the indentation causes the observation portion to form a lower cutting section and an upper shallow section; and the indentation causes the opening of the through hole to form a lower edge and an upper edge.

8. The spinal endoscope assembly as claimed in claim 6, wherein the second end of the conduit, designed for insertion into the fitting portion of the transparent tube, is further cut with a positioning groove; an inner wall of the fitting portion is provided with a coupling rib at a location corresponding to the lower positioning groove; when the transparent tube is sleeved around the conduit via the fitting portion, the lower positioning groove is aligned with and engaged with the coupling rib; and an engagement between the lower positioning groove and the coupling rib secures a combination between the conduit and the transparent tube.

9. The spinal endoscope assembly as claimed in claim 6, wherein the first end of the conduit, designed for insertion into the upper holder, is further cut with an upper positioning groove; an inner wall of the perforation in the upper holder is provided with a coupling rib at a location corresponding to the upper positioning groove; when the conduit is inserted into the upper holder, the upper positioning groove is aligned with and engaged with the coupling rib; and an engagement between the upper positioning groove and the coupling rib secures a combination between the upper holder and the conduit.

10. The spinal endoscope assembly as claimed in claim 6, wherein an outer side of the upper holder is further extended with two grip handles; the two grip handles extend in opposite directions; and one of the grip handles has an end with a groove recessed into a top surface thereof.

11. The spinal endoscope assembly as claimed in claim 6, wherein the endoscope tube further includes a main tube and an end cap; one end of the main tube is connected to the gun body; one end of the end cap is attached to the other end of the main tube; an end surface at the other end of the end cap is designed as a slanted face; and one end of the camera module is exposed from and aligned with the slanted face.

12. The spinal endoscope assembly as claimed in claim 11, wherein the camera module further includes a lens and a chip; the chip is located inside the main tube; the lens is positioned inside the end cap and is electrically connected to the chip; and the chip is electrically connected to an external electronic component, either wirelessly or via a wired connection, to transmit data captured by the lens.

13. The spinal endoscope assembly as claimed in claim 11, wherein a main axis is defined relative to the slanted face, and an angle between the slanted face and the main axis ranges from 10 degrees to 35 degrees.

14. The spinal endoscope assembly as claimed in claim 12, wherein at least one light emitting element is further disposed inside of the end cap and electrically connected to the chip.

15. The spinal endoscope assembly as claimed in claim 6, wherein an outer casing is further sleeved around the gun body; the outer casing includes a first sleeve and two handle sleeves; the first sleeve is sleeved around an outer surface of the gun body and is equipped with an elastic buckle that detachably engage with any of a plurality of grooves on the outer surface of the gun body; the first sleeve is equipped with a plurality of connecting columns on an end opposite the elastic buckle; the two handle sleeves are U-shaped and each has two ends detachably inserted into the assembly grooves of the connecting columns; and a clamping area is formed between the two handle sleeves.
